(19) United States
(12) Patent Application Publication
(10) Pub. No.: US 2005/0010286 A1
(43) Pub. Date: Jan. 13, 2005

(54) Heart failure mitral annuloplasty ring with removable central posterior portion

(75) Inventor: Venkataramana Vijay, Tarrytown, NY (US)

Correspondence Address: GORDON & JACOBSON, P.C., 65 WOODS END ROAD, STAMFORD, CT, 06905, US

(73) Assignee: Vedic Biotechnology, Inc.

(21) Appl. No.: 10/617,610

(22) Filed: Jul. 11, 2003

(51) Int. Cl. A61F 2/24

(52) U.S. Cl. 623/2.36

(57) ABSTRACT

An annuloplasty ring includes an anterior portion, and a posterior portion which defines a central portion and two lateral portions. The ring is adapted for optional removal of the anterior and/or the central posterior portion. Removal of the central posterior portion reduces the gradient across the ring providing enhanced valve performance. Removal of the anterior portion preserves normal annular movement. The lateral posterior portions are stiffer than the construction at the anterior and central posterior portions. If the ring is used with the central posterior portion intact, the gradient is also reduced. The ring includes indicia of multiple sets of suture markings, each set identifying a plurality of suture locations about the perimeter of the ring which are adapted to cinch the annulus a predetermined amount about the ring. A single ring may be used to cinch the annulus in accord with relatively different degrees of desired valve area reduction.

### BACKGROUND OF THE INVENTION

1. Field of the Invention

This invention relates broadly to implantable prostheses. More particularly, this invention relates to annuloplasty rings specifically adapted for the mitral valve of the heart.

2. State of the Art

Mitral regurgitation is a “leaking” of the mitral valve which connects the left atrium and the left ventricle of the heart. When the left ventricle contracts to eject blood to the rest of the body, the mitral valve closes to prevent blood from passing in the wrong direction; i.e., into the left atrium. When the mitral valve fails to close properly and mitral regurgitation (MR) develops. If the MR is severe, mitral valve repair or replacement is needed to preserve the function of the left ventricle and to prevent congestive heart failure from developing. Mitral valve repair is often done to eliminate MR and prevent the necessity of mitral valve replacement.

During mitral valve repair, a portion of the redundant valve tissue is resected and the valve leaflets are reshaped to eliminate MR. In degenerative disease of the mitral valve leaflets, the annulus about the leaflets typically increases by approximately one hundred to two hundred percent. In such case, an annuloplasty ring is provided at the annulus and the annulus is sewn to the ring to create a purse string effect around the base of the valve which helps the leaflets meet when the valve closes. This also restores the anatomical size and shape of the valve and supports the repaired mitral valve to prevent recurrent dilatation. Due to the excess leaflet tissue caused by degenerative disease, any size mismatching of the annuloplasty ring and the mitral annulus is of little consequence.

However, in heart failure, the leaflets are not enlarged. Thus, choosing the appropriate size for an annuloplasty ring is critical to avoid the occurrence of MR from continuing dilatation of the heart.

Each of the anterior and posterior leaflets of the annulus is divided by nomenclature into thirds. The anterior leaflet has a leftmost portion A₁, a central portion A₂, and a rightmost portion A₃. Similarly, the posterior anterior leaflet has a leftmost portion P₁, a central portion P₂, and a rightmost portion P₃. early leakage of the mitral valve in heart failure starts at two specific locations, namely P₁ and P₃. However, P₂ is the portion directly in the path of blood from the left atrium to the ventricle.

It has been noted by the present inventor that prior art mitral annuloplasty rings effect an undesirable gradient across the mitral valve which may cause a backflow of blood into the lungs. Prior art mitral annuloplasty rings remodel the annulus by providing a 3:4 ratio between the anteroposterior and transverse diameters of a normal mitral valve for what is generally considered optimal hemodynamic performance. In addition, the outer cross-sectional diameter of a state of the art ring is relatively uniform about its circumference.

Annuloplasty rings are typically made of flexible polymers and generally are available in ring-shaped (annular) or C-shaped configurations. The C-shaped designs include a posterior portion (including substantially transverse lateral portions and a central portion therebetween), but no anterior portion, which operates to effect a reduced gradient (but does not eliminate the gradient). In addition, some annuloplasty rings, e.g., the Sulzer Carbomedics AnnuloFlex™ ring and the St. Jude Medical Tailor™ ring, have a ring-shaped configuration that is adapted to be converted into a C-shaped configuration by removal of the anterior portion of the ring. Annuloplasty rings generally also include commissure guides (or trigone markings) by which to reference a ring relative to the left and right valve leaflet commissures (or left and right fibrous trigones) and the posterior midline of the valve annulus to facilitate implantation.

Annuloplasty rings are also available in a variety of sizes permitting selection of a ring which most appropriately corresponds to the intended size of the post-operative annulus. However, this requires that a medical care facility stock each of the variety of sizes, thereby complicating inventory control. Each size of ring includes thereon, or has associated therewith a guide which includes, markings indicating spaced-apart locations for a set of suture ties so that the ring can be coupled to the mitral valve annulus.

### SUMMARY OF THE INVENTION

It is therefore an object of the invention to provide an annuloplasty ring that can produce multiple degrees of valve area reduction by having spaced-apart markings producing different degrees of reduction of the annulus, thereby obviating the need to stock as many sizes of rings as in the prior art.

It is another object of the invention to provide an annuloplasty ring which provides desirable hemodynamic performance.

It is a further object of the invention to provide an annuloplasty ring which reduces a gradient across the valve to physiological levels.

It is also an object of the invention to provide an annuloplasty ring which can be used in a ring-shaped configuration, a C-shaped configuration, and other configurations most suitable to treat mitral regurgitation.

In accord with these objects, which will be discussed in detail below, an annular mitral annuloplasty ring includes an anterior portion and a posterior portion having central and substantially transverse lateral portions. Alternatively, the ring may be C-shaped and formed without the entirety of, or a portion of, the anterior portion.

Regardless of whether the ring is completely annular or C-shaped, according to a first preferred aspect of the invention, the ring includes a posterior portion defining a central portion and two lateral portions. The ring is adapted in construction for stabilization and non-reduction of the central posterior portion, while significant reduction of lateral portions is facilitated. It has been determined by the inventor that, in many cases, reduction of the central posterior portion of the ring results in an increased gradient. Therefore, the ring of the invention does not reduce, but only stabilizes the central portion of posterior leaflet, and consequently decreases the gradient across the valve relative to prior art rings which cinch a central posterior portion of the valve annulus.

According to a second preferred aspect of the invention, the construction of the ring at the lateral posterior portion is different than the construction at the central posterior portion (i.e., the portion adapted to optionally be removed). The lateral posterior portions are substantially stiffer than the central posterior portion. A softer central posterior portion minimizes a gradient where the central posterior portion remains integral with the ring, while the lateral posterior portions contribute strength and competence of the valve during closure of the leaflets. One preferred manner of effecting stiffer lateral posterior portions is to construct the sides as relatively flatter than a more tubular central portion.

From the foregoing, it is appreciated that the mitral annuloplasty ring of the invention is hemodynamically optimized to reduce a gradient thereacross, and improve competence of the valve leaflets by selectively reducing the lateral posterior portions.

According to a third preferred aspect of the invention, the ring includes indicia of multiple sets of suture markings, each set identifying a plurality of suture locations about the perimeter of the ring which are adapted to cinch the annulus by a predetermined amount about the ring. Thus, a single ring may be used to cinch the annulus in accord with relatively different degrees of desired valve area reduction. This is in contrast to the prior art, where multiple rings of different dimensions are required for the same effect. Thus, each ring of the invention corresponds to multiple rings of different sizes and reduction capabilities of the prior art.

Additional objects and advantages of the invention will become apparent to those skilled in the art upon reference to the detailed description taken in conjunction with the provided figures.

### BRIEF DESCRIPTION OF THE DRAWINGS

FIG. 1 is a plan view of an mitral annuloplasty ring according to the invention;

FIG. 2 is a cross-section across line 2-2 in FIG. 1;

FIG. 3 is a cross-section across line 3-3 in FIG. 1;

FIG. 4 is a cross-section across line 4-4 in FIG. 1;

FIG. 5 illustrates the mitral annuloplasty ring of the invention shown implanted, where both the anterior and posterior portions of the ring are used;

FIG. 6 illustrates the mitral annuloplasty ring of the invention shown implanted, where the anterior portion of the ring is removed;

FIG. 7 illustrates the mitral annuloplasty ring of the invention shown implanted, where both the anterior portion and central posterior portions of the ring are removed, leaving only the lateral posterior portions of the ring implanted at the valve;

FIG. 8 is a second embodiment of a mitral valve annuloplasty ring according to the invention; and

FIG. 9 is an embodiment of a instrument which includes suture guides in accord with the invention.

### DETAILED DESCRIPTION OF THE PREFERRED EMBODIMENTS

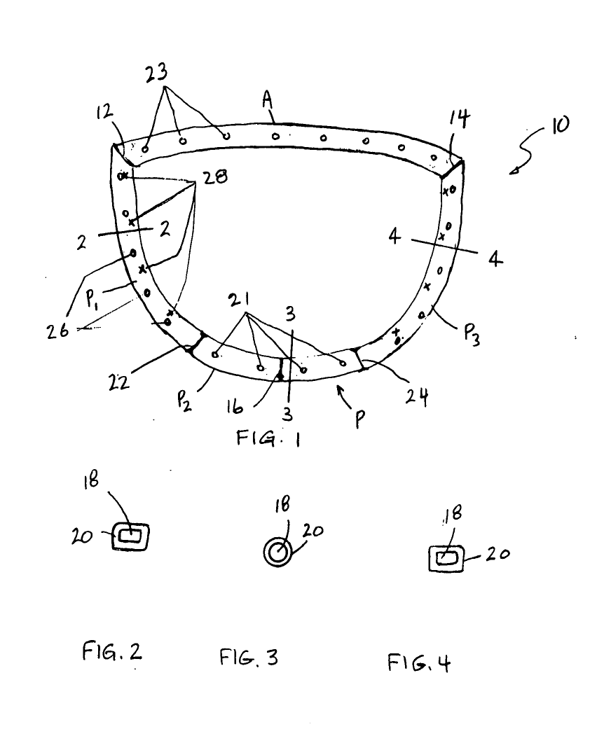

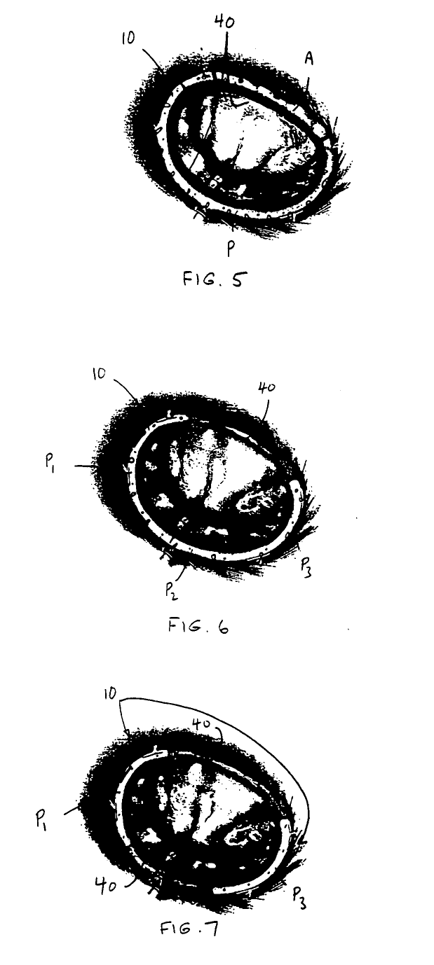

Turning now to FIG. 1, a mitral annuloplasty ring 10 is shown. The ring 10 includes a shallowly curved anterior portion A, and a steeper curved posterior portion P. The ring is preferably provided with trigone guides 12, 14 (or alternatively commissure guides) and optionally a posterior midline guide 16 which together facilitate alignment of the ring relative to anatomical landmarks of the mitral valve. Referring to FIGS. 2 through 4, the ring 10 is preferably constructed of an inner structural constituent 18, e.g., resilient polytetrafluoroethylene (PTFE), which is surrounded by a fabric outer layer 20 through which suture needles and suture can be passed to secure the ring at the valve annulus. Other materials known in the art can also be used in the alternative or in combination with the above described materials.

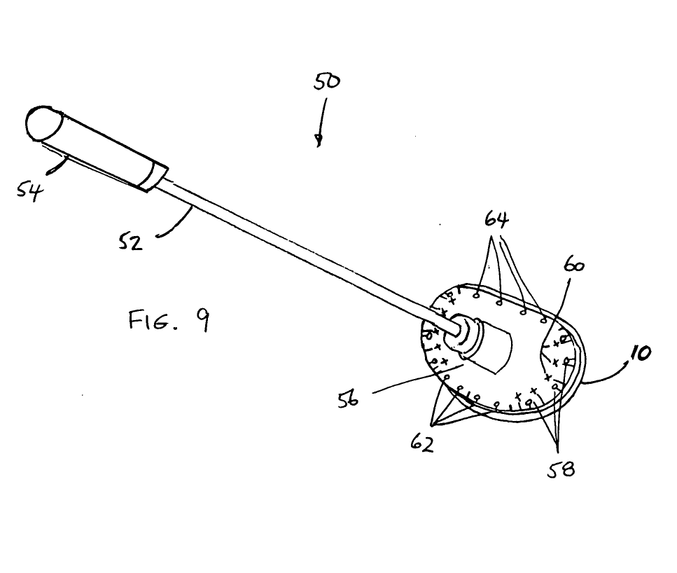

According to a first preferred aspect of the invention, the posterior portion P includes a central portion P₂ and substantially transverse lateral portions P₁ and P₃ on either side of the central portion. The ring 10 is preferably adapted in construction for optional removal of the central posterior section P₂, preferably after implantation of the ring at the valve (See FIG. 7). That is, the ring 10 at the junction of P₁ and P₂ and junction of P₂ and P₃ preferably includes indicia 22, 24 indicating where a blade may be used to cut the ring and/or is provided with a weakened section (e.g., reduced diameter), or even a discontinuity, of the structural constituent 18 at the indicated locations 22, 24 to facilitate cutting and removal of the central posterior portion P₂. If removal of the central portion P₂ is performed, it is preferably performed after suturing the lateral posterior portions P₁ and P₃ at the valve annulus. It has been determined by the inventor that, in many cases, the central posterior portion P₂ of the ring 10 is not required to abate MR or support the annulus and may, in fact, contribute to an excessive gradient across the ring 10. By eliminating the central posterior portion P₂, the gradient is reduced relative to prior art to thereby provide superior results.

It has also been determined by the inventor that, in many cases, reduction of the P₂ of the valve annulus contributes to an excessive gradient across the ring 10. The P₂ portion of the ring 10 includes suture markings 21 (represented by circles) which are spaced so as to effect no annular reduction if the P₂ portion of the ring is kept intact and coupled to the valve. By not reducing the central posterior portion P₂, the gradient is reduced relative to prior art to thereby provide superior results. In addition, similarly spaced-apart markings 23 (also represented by circles) between indicia 12 and 14 (FIG. 1) of the anterior leaflet are provided so as to not effect reduction of the anterior annulus.

Referring to FIGS. 2 through 4, and according to a second preferred aspect of the invention, the construction of the ring at the lateral posterior portions P₁ and P₃ is different than the construction at the central posterior portion P₂. The lateral posterior portions P₁, P₃ are slightly stiffer than the central posterior portion P₂. One preferred manner of effecting stiffer lateral portions P₁ and P₃ is to construct the sides relatively flatter, and the central posterior portion P₂ more cylindrical. That is, the lateral posterior portions P₁ and P₃ preferably have a smaller dimension in the direction of blood flow and a relative greater dimension transverse to the direction of blood flow. The more flexible central posterior portion P₂ minimizes a gradient where the central posterior portion remains integral with the ring after implantation. In addition, the lateral posterior portions P₁, P₃ contribute strength, but do not significantly affect the gradient. The similarly structured more flexible anterior portion allows preservation of normal annular movement during the cardiac cycle.

From the foregoing, it is appreciated that the mitral annuloplasty ring of the invention is hemodynamically optimized to reduce a gradient thereacross.

Referring back to FIG. 1, according to a third preferred aspect of the invention, the ring 10 includes multiple circumferential sets 26, 28 of indicia (where only a subset of each set of indicia is identified by the reference numerals) for suture placement. FIG. 1 distinguishes the sets of indicia based upon a discrete shape (e.g., circles 26 and cruciforms 28) for ease of distinction in the black and white drawing. However, distinctions based upon discretely colored markings (e.g., colored sutures extending circumferentially about the ring) or other visual indicators may be preferred. Each marking within a set 26, 28 is preferably spaced apart from another marking of the same set by a predetermined distance (e.g., 2.5 mm or 3.0 mm or similar increments). Each set 26, 28 of indicia thusly corresponds to a predetermined amount of cinching about the ring 10. The physician selects one of the plurality of sets of markings according to the degree by which the physician assesses that the valve annulus should be cinched. Thus, a single ring may be used to cinch the annulus in accord with relatively different degrees of desired valve area reduction. In contrast, the prior art would require different rings each optimized for a different size of reduction.

Alternatively, the indicia corresponding to multiple sets of suture locations sizes may be provided to instrumentation, such as a ring holder to thereby guide the surgeon to the same effect. For example, instrument 50 includes a handle 52 having a manual gripping element 54 at one end and a ring holder 56 removably coupled at its other end. Such ring holders are well known in the art. In accord with the invention, the ring holder 56 is coupled to a ring 10, e.g., with sutures (not shown), and includes multiple sets of suture guides 58 (circles), 60 (cruciforms) along portions of the holder 10 which correspond to the P₁ and P₃ portions of the ring 10. The portions of the holder 10 which correspond to the P₃ and anterior portions of the ring 10 are each preferably provided with a single set of suture guides 62 (along P₃) and 64 (along the anterior portion).

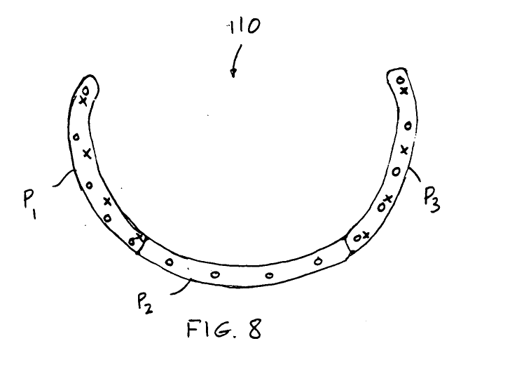

An annuloplasty ring 10 according to the invention may be implanted in any of three configurations at the mitral valve. Referring to FIG. 5, in accord with the a first method of implantation, the valve annulus 40 is sutured to both the anterior and posterior portions A and P of the ring 10. Thus, the ring 10 is circumferentially continuous (with the anterior portion A intact) in its implanted state. Referring to FIG. 6, in a second method of implantation, the valve annulus 40 is sutured to the posterior portions P₁, P₂ and P₃ of the ring 10, and the anterior portion of the ring is removed from the implant, e.g., by cutting. While the central posterior portion P₂ remains intact, the structural design of this portion operates to limit the gradient across the anterior portion of the valve. Referring to FIG. 7, in a third method of implantation, the valve annulus is sutured to the lateral posterior portions P₁ and P₃ of the ring, but not the central posterior portion P₂ or the anterior portion A. The central posterior portion P₂ and anterior portion A are then removed from the ring after the valve annulus is secured to the lateral posterior portions P₁ and P₃. As the ring is structurally stiffer along the lateral posterior portions, the annulus is nevertheless stably supported. Moreover, removal of the central posterior portion P₂ greatly reduces the gradient across the valve and provides a superior result relative to prior art annuloplasty rings. Thus, the invention includes a method whereby the lateral posterior portions of an annulus are supported by an implant, but the anterior and central posterior portion of the annulus are unsupported by an implant so as to reduce a gradient across the mitral valve.

Turning now to FIG. 8, another embodiment of an annuloplasty ring according the invention is shown. The ring 110 is C-shaped and formed without a significant portion of the anterior portion A or even the entirety thereof. Preferably, all other features of ring 10, e.g., a construction permitting removal of central portion P₂ and a plurality of sutures sets, are incorporated into ring 110. The ring may be implanted in accord with the methods described with respect to FIGS. 6 and 7.

There have been described and illustrated herein embodiments of an annuloplasty mitral valve ring and a method of annuloplasty. While particular embodiments of the invention have been described, it is not intended that the invention be limited thereto, as it is intended that the invention be as broad in scope as the art will allow and that the specification be read likewise. It will therefore be appreciated by those skilled in the art that yet other modifications could be made to the provided invention without deviating from its spirit and scope as claimed.

1. A mitral annuloplasty device, comprising:
a ring-shaped structural component sized for mitral valve annuloplasty, said structural component having a relatively shallowly curved anterior portion and a relatively steeper curved C-shaped posterior portion including a central portion and first and second lateral portions,
wherein said posterior portion includes demarcations between said first lateral portion and said central portion and between said central portion and said second lateral portion facilitating removal of said central portion from between said first and second lateral portions.

2. An annuloplasty device according to claim 1, wherein:
said demarcations are structural.

3. An annuloplasty device according to claim 2, further comprising:
visual indicia coincident with said structural demarcations.

4. An annuloplasty device according to claim 3, wherein:
said structural component is provided with a relatively softer outer layer, and said visual indicia is provided on said outer layer.

5. An annuloplasty device according to claim 3, wherein:
said first and second lateral portions each include one of a trigone marking and a commissure marking.

6. An annuloplasty device according to claim 1, wherein:
said lateral portions are oriented substantially transverse to said central portion.

7. An annuloplasty device according to claim 1, wherein:
said central and first and second lateral portions define a plane, and said first and second lateral portions are relatively stiffer than said central portion in a direction transverse to said plane.

8. An annuloplasty device according to claim 1, wherein:
said central portion defines a cross-sectional shape that is rounder than a cross-sectional shape defined by said first and second lateral portions.

9. An annuloplasty device according to claim 1, further comprising:
means for identifying sets of suturing locations on said device, each said set corresponding to a discrete predetermined amount of cinching of an annulus of a mitral valve.

10. An annuloplasty device according to claim 9, wherein:
said means for identifying includes visual indicia.

11. An annuloplasty device according to claim 10, wherein:
said visual indicia includes sets of visual indicia distinguished by at least one of color and shape.

12. An annuloplasty device according to claim 11, wherein:
said means for identifying includes discrete indicia corresponding to each of said sets of suturing locations, and said indicia corresponding to each of said sets are spaced apart from each other by a distance different than a distance by which indicia in the other of said sets is spaced apart.

13. A mitral annuloplasty device, comprising:
a C-shaped structural component sized for mitral valve annuloplasty, said structural component including a central portion and first and second lateral portions,
wherein said structural component includes demarcations between said first lateral portion and said central portion and between said central portion and said second lateral portion facilitating removal of said central portion from between said first and second lateral portions.

14. An annuloplasty device according to claim 13, wherein:
said demarcations are structural.

15. An annuloplasty device according to claim 14, further comprising:
visual indicia coincident with said structural demarcations.

16. An annuloplasty device according to claim 15, wherein:
said structural component is provided with a relatively softer outer layer, and said visual indicia is provided on said outer layer.

17. An annuloplasty device according to claim 15, wherein:
said first and second lateral portions each include one of a trigone marking and a commissure marking.

18. An annuloplasty device according to claim 13, wherein:
said lateral portions are oriented substantially transverse to said central portion.

19. An annuloplasty device according to claim 13, wherein:
said central and first and second lateral portions define a plane, and said first and second lateral portions are relatively stiffer than said central portion in a direction transverse to said plane.

20. An annuloplasty ring according to claim 13, wherein:
said structural component further includes an anterior portion anteriorly coupling said first and second lateral portions, such that said ring is annular in shape.

21. An annuloplasty device according to claim 13, wherein:
said central portion defines a cross-sectional shape that is rounder than a cross-sectional shape defined by said first and second lateral portions.

22. An annuloplasty device according to claim 13, further comprising:
means for identifying sets of suturing locations on said device, each said set corresponding to a discrete predetermined amount of cinching of an annulus of a mitral valve.

23. An annuloplasty device according to claim 22, wherein:
said means for identifying includes visual indicia.

24. An annuloplasty device according to claim 23, wherein:
said visual indicia includes sets of visual indicia distinguished by at least one of color and shape.

25. An annuloplasty device according to claim 22, wherein:
said means for identifying includes discrete indicia corresponding to each of said sets of suturing locations, and said indicia corresponding to each of said sets are spaced apart from each other by a distance different than a distance by which indicia in the other of said sets is spaced apart.

26. A method for mitral valve annuloplasty, comprising:
a) providing an annuloplasty device having a central posterior portion and first and second lateral posterior portions;
b) coupling lateral posterior portions of an annulus of the mitral valve to the first and second lateral posterior portions of the annuloplasty device; and
c) removing the central posterior portion of the annuloplasty device while maintaining the coupling between the lateral posterior portions of the annulus of the mitral valve and the first and second lateral posterior portions of the annuloplasty device.

27. A method according to claim 26, wherein:
said providing includes providing an annuloplasty device in which said first and second lateral posterior portions are at least one of stiffer and thicker than said central posterior portion.
